(19) United States
(12) Patent Application Publication
(10) Pub. No.: US 2003/0003457 A1
(43) Pub. Date: Jan. 2, 2003

(54) Bio-polymer array system with detection sensitivity enhanced by radiation treatment

(76) Inventor: Valeri Golovlev, Oak Ridge, TN (US)

Correspondence Address: Valeri V. Golovlev, 107 Canterbury Rd., Oak Ridge, TN, 37830, US

(21) Appl. No.: 09/891,421

(22) Filed: Jun. 26, 2001

(51) Int. Cl. C12Q 1/68; C12M 1/34

(52) U.S. Cl. 435/6; 435/287.2

(57) ABSTRACT

Devices and techniques are disclosed for sequencing, fingerprinting, or mapping bio-polymer molecules in micro-array format by tagging molecules with radiation absorbing particles and exposing tagged molecules to electromagnetic radiation such as microwave radiation. The use of radiation absorbing material for tagging enhances detection sensitivity by dissipating energy of the radiation in spots on surface where tagged molecules are located. Proposed system can be particularly beneficial when used as a reader system for DNA and protein microarrays in genomic and proteomic applications, for reading affinity assays, and for detection of a trace amount of chemical or biological species of interest on a surface.

### BACKGROUND OF THE INVENTION

The present invention relates to the field of bio-polymer analysis, detection, and sequencing which are of interest in biomedical, biological, and chemical research and applications. More specifically, different embodiments of the invention providing improved techniques for analyzing arrays of nucleic acids, hybridizing nucleic acids, detecting mismatches in a double-stranded nucleic acid composed of a single-stranded probe and a target nucleic acid, and determining the sequence of DNA or RNA or other bio-polymers.

Nucleic acid hybridization has become an increasingly important route for DNA sequencing and gene expression studies. Recently developed combinatorial DNA chips, which rely on the specific hybridization of target and probe DNA on a solid surface, attracted tremendous interest among biologists. A historical background as well as a description of the basic concept of bio-polymer arrays for study and diagnostics of biological systems is provided in the following references:

A. M. Maxam and W. Gilbert, “A New Method for Sequencing DNA”, Proc. Natl. Acad. Sci. U.S.A., 74, 560-564 (1977)

Saiki et al, “Genetic Analysis of Amplified DNA with Immobilized Sequence-Specific Oligonucleotide Probes”, Proc. Natl. Acad. Sci. USA., 86, 6230-6234 (1989)

Chee et al, “Accessing Genetic Information With High-Density DNA Arrays”, Science, 274, 5287 (1996)

Pastinen et al, “Minisequencing: A Specific Tool for DNA Analysis and Diagnostics on Oligonucleotide Arrays”, Genome Research, 7, 606-614 (1997)

P. A. Fodor, “Techwire”, Science, 277, 5324 (1998)

Landegren et al, “Reading Bits of genetic Information: Methods for Single-Nucleotide Polymorphism Analysis”, Genome Research, 8, 769-776 (1998)

Cho et al, “Parallel Analysis of Genetic Selections Using Whole Genome Oligonucleotide Arrays”, Proc. Natl. Acad. Sci. USA., 95, 3752-3757 (1998)

Kricka et al., “Miniaturization of Analytical Sytems”, Clinical Chemistry, 44:9, 2008-2014 (1998)

Southern et al, “Molecular Interactions on Microarrays”, Nature Genetics, 21(1), 5-10 (1999)

Duggan et al, “Expression Profiling Using cDNA Microarrays”, Nature Genetics, 21(1), 10-15 (1999)

Cheung et al, “Making and Reading Microarrays”, Nature Genetics, 21(1), 15-20 (1999)

Lipshutz et al, “High Density Synthetic Oligonucleotide Arrays”, Nature Genetics, 21(1), 20-25 (1999)

H. Ge, “UPA, a Universal Protein Array System For Quantitative Detection of Protein-Protein, Protein-DNA, Protein-RNA and Protein-Ligand Interactions”, Nucleic Acids Research, 28(2), e3 (2000)

G. MacBeath, S. L. Schreiber, “Printing Proteins As Microarrays for High-Throughput Function Determination”, Science, 289, 1760 (2000)

see also

Adelman, (1997), U.S. Pat. No. 5,656,429

Hollis et al., (1998), U.S. Pat. No. 5,846,708

Wang et al., (1999), U.S. Pat. No. 5,922,617

Dale et al., (2000), U.S. Pat. No. 6,087,112;

Fodor (2001), U.S. Pat. No. 6,197,326;

Hori et al, (2001), U.S. Pat. No. 6,194,148;

Virtanen, (2001), U.S. Pat. No. 6,200,755

Schwartz et al., (2001), U.S. Pat. No. 6,221,592

Fodor et al., ((1992), Foreign Pat. No. WO 92/10588

Virtanen, (1998), Foreign Pat. No. WO 98/01533

Ribi, (1990), Foreign Pat. No. EP 0 402 917

and references herein.

An attractive feature of the microarray technology for genomic applications is that microarrays have the potential to monitor the whole genome on a single chip, so that researchers can have a complete picture of the interaction among thousands of genes simultaneously. Possible applications of DNA microarrays include gene discovery, disease diagnosis, drug discovery, toxicological research, and micro-organisms detection/characterization. Fast growing applications of microarrays put new demands for improving detection sensitivity of hybridized complexes on the surface of DNA chips. Currently, the most common approach to detect DNA bound to the microarray is to label them with a reporter molecule that identifies DNA presence. The reporter molecules emit detectable light when excited by an external light source. Light emitted by a reporter molecule has a characteristic wavelength, which is different from the wavelength of the excitation light, and therefore a detector such as Charge-Coupled Device (CCD) or a confocal microscope can selectively detect a reporter's emission. Most commercial systems required 10⁷ or more dye-tagged DNA molecules for reliable detection, mostly due to limitation of sensitivity by a background from scattered light of the excitation source. Another important issue which limits the detection sensitivity is the relatively low number of reporter groups which can be attached to a single DNA (usually not more than one reporter molecule per 20-100 bases of DNA). To overcome this limitation, the use of nanoparticle probes in combination with optical detection has been proposed by Taton et al, “Scanometric DNA Array Detection with Nanoparticle Probes”, Science, 289, 1757-1760 (2000), see also Storhoff et al, “One-Pot Colorimetric Differentiation of Polynucleotides with Single Base Imperfections Using Gold Nanoparticle Probes”, J. Am. Chem. Soc., 120, 1959-1964 (1998). Gold nanoparticles with oligonucleotides attached to their surface were used to indicate the hybridization of a DNA on a transparent substrate. The gain of detection sensitivity can be explained by the fact that the amount of tagging material in a single nanoparticle is a few order of magnitude higher than the amount of material delivered by a single reporter molecule. Furthermore, to facilitate the visualization of a labeled nanoparticle hybridized to the array surface, a signal amplification method can be used in which silver ions are reduced by hydroquinone to silver metal at the surface of the gold nanoparticles. Such amplification technique has previously been used to visualize protein, antibody-, and DNA-conjugated gold nanoparticles in histochemicals electron microscopy studies. When applied to microarrays, this amplification technique enables very low surface coverage of nanoparticle probes to be visualized by a simple flatbed scanner or naked eye. However, the additional chemical treatment of the surface with hybridized species for amplification also imposes significant risk of losing analytes due to undesirable wash away, chemical decomposition, and creating background by non-specific release of silver on the microarray surface. Therefore, development of new techniques for amplification of the presence of metal particles on a surface, without the disadvantages described in the above, is of great interest for microarray technologies for DNA and protein applications. It can also be applied to other fields where measurement of a small amount of materials on a surface is required.

### SUMMARY OF THE INVENTION

In this invention, devices and techniques are disclosed for qualitative and quantitative characterization of bio-polymers, such as oligonucleotides and proteins, for the purpose of sequencing, fingerprinting, or mapping said bio-polymers. The approach we propose is comprising the steps of:

a) preparing a surface on which analysis will be performed by covering a solid substrate with a thin layer of material, also referred to as sensitive layer, with distinguishable properties such as mechanical, optical, magnetic, or chemical property;

b) preparing multiple test sides on said surface by immobilizing probes. The probes will be of various known structures, selected to bind with molecular structures, such as biological polymers, which may be in the sample of being analyzed. Location and type of each particular probe on the surface is known and therefore probe location on the surface can be used to identify type/sequence of the probe;

c) hybridizing a target bio-polymer molecule with a bio-polymer probe on said surface, which said probe is complementary to a region of the target molecule;

d) labeling/tagging the hybridized probe-target complexes with a metal particles or particles of other material capable, if present, to absorb and dissipate electromagnetic radiation including microwave radiation, infra-red radiation or visible, or, equally acceptable, UV light, all of which are referred herein by term “electromagnetic radiation” or “EM radiation”;

e) treating, when necessary, the substrate with immobilized tagging particles by confining said substrate between two smooth solid surfaces (also referred as casts) and by applying pressure to the casts of usually not less than 10⁵ Pa, which said pressure is of capable to squeeze said substrate and the tagging particles on said substrate surface to the degree when the tagging particles start to penetrate or immerse into the sensitive layer of said substrate; treatment, as disclosed herein, can improve the mechanical contact between tagging particles and solid substrate;

f) exposing said surface with labeled probe-target complexes by electromagnetic radiation under conditions where the energy of EM radiation can be dissipated by said tagging particles; dissipation of radiation energy by tagging particles causes change of local properties of said sensitive layer on said surface in spots where probe-target complexes are located;

g) analyzing said surface by measuring property of the sensitive layer, using appropriate technique for measuring mechanical, optical, magnetic, or chemical parameters of said sensitive layer as known from prior art, see. Deviation or change of the quantitative characteristic of the sensitive layer resulting from EM radiation exposure would point to the spots where probes were hybridized to the targets, therefore pointing to the presence of targets with a sequence complimentary to the sequence of corresponding known probe(s);

The use of EM radiation absorbing material for tagging enables to enhance sensitivity of the array reading system. Indeed, dissipation of energy of electromagnetic radiation by tagging particles produces marks on the surface due to effects, such as, but are not limited to, overheating, surface arcing, and/or ablation (micro-explosion) on the surface. Said effects can result in change of surface property in spots covered by tagging particles and thus can make more “visible” the small amount of reporter material immobilized on a surface by hybridization events. Said tagging particles can include, but are not limited to, a small size of metal particles. Said surface property might include, but is not limited to, optical properties, mechanical properties, magnetic properties, or chemical properties of the surface. Array analysis can be performed using devices similar to computer floppy drive or digital CD drive. Proposed system can be particularly beneficial when used as a reader system for DNA and protein microarrays in genomic and proteomic applications, for reading affinity assays, and for detection of a trace amount of chemical or biological species of interest on a surface. The method can also be used for making readable records of digital data for computer applications and for making audio, and video records.

### BRIEF DESCRIPTION OF DRAWINGS

Other objects, features and advantages of the present invention will become apparent from the detailed description of the preferred embodiments of the invention which follows, when considered in light of the accompanying drawing in which:

FIG. 1 shows the First step of preparing media for monitoring analytes on a surface. A solid surface, on which analysis will be performed, is covered or painted with a thin layer of a material with a distinguishable property, i.e., either optical property, mechanical property, magnetic property, or chemical property. Said substrate might be a plate from a glass, plastic, or any other material which does not have significant absorption of electromagnetic radiation, which said radiation is described herein. Said substrate might have any shape and size including, but not limited to, the rectangular shape or might be shaped as a disk, similar to a computer compact disk (CD) or computer floppy disk.

FIG. 2 shows preparation of a microarray of probe oligonucleotides or proteins on the surface of the substrate as described in FIG. 1. Location on the surface of each specific type of probe is known and the location of the probe can be used to uniquely identify the type of the probe, for example, based on its sequence in the case of oligonucleotides, or based on sequence and/or structure in the case of proteins.

FIG. 3 shows the step when said substrate surface with immobilized probes is exposed to the solution of the target (i.e., proteins or oligonucleotides, depending on the type of analysis performed), which would be bound or hybridized to the probes on the surface, if the said target species are complementary to the probes because of the primary sequence, structural or chemical properties as shown for (A) and (C) species. The target species do not bind to the surface, as it is shown for the specie (B), if the target is not complementary to the probes on the surface. The target species have the capability to attach reporter molecules or particles through the reaction similar to biotin-streptavidin reaction, through the formation of thioether bond, through absorbing thiol-functionalized molecules on gold surface, or by using other types of binding known from the previous art.

FIG. 4 shows the substrate surface after it was exposed to reporter material. Said exposure to reporter material can be pursued either using liquid solution, or powder of the reporter material, or by applying reporter species from a gas phase. The spots on the surface where target and probe were hybridized would be immobilized by reporter material. Next, the substrate is exposed to EM radiation, such as, for example, microwave radiation. Extensive release of radiation energy in the spots where the reporter material is bound to the surface modifies or damages the underlying layer of the substrate, which was prepared as described and depicted in FIG. 1. The size of the area where the substrate coverage is affected by EM radiation might be significantly bigger than the area originally covered by a reporter material. This, in fact, increases or amplifies the effect from presence of the small amount of reporter material on the surface and makes it possible to detect the location on the surface and/or measure the quantity of the reporter material. Since the reporter material is presented only in spots where probe and target were bound or hybridized, the modification of the substrate's surface by microwave radiation can be used to find which probes are complimentary to the targets. Therefore, a target sequence or structural information can be obtained for DNA and protein microarrays respectively.

Yet in another possible design of the measurement system, the presence of the specie of interest can reduce or block the attachment of reporter material to the surface, such that said specie can be detected because of the absence, not presence, of the reporter material at a particular location on the surface.

FIG. 5 shows the step when the substrate with immobilized tagging particles is confined between two smooth solid surfaces and pressure, P, is applied to caste said substrate such that tagging particles on the substrate surface are partially or completely penetrated or immersed into the sensitive layer of said substrate; such treatment, although is optional, can provide better mechanical contact of the tagging particles and the microarray substrate surface.

FIG. 6 shows the step when the substrate with reporter material attached to the surface is exposed to EM radiation. Extensive release energy of EM radiation in the spots where the reporter material is bound to the surface causes modification or damage of the substrate surface, which first was prepared as depicted in FIG. 1.

FIG. 7 illustrates the effect of enhancing detection sensitivity by monitoring radiation damage on the substrate surface. The size of the area where the substrate is affected by microwave radiation might be significantly bigger than the area originally covered by a reporter material. This effect increases or amplifies the presence of the small amount of reporter material on the surface and makes it possible to detect location on the surface and measure quantity of the reporter material.

FIG. 8: In the another embodiment of the invention, the substrate which will be used for detection can be covered or painted by a thin layer of a magnetic paint, similar to that used in manufacturing magnetic recording media. Magnetic pattern on the surface of the substrate is recorded for later use as a reference. Substrate surface is exposed to solution of reporter particles which are immobilized on surface in spots where probes and targets are hybridized (see FIGS. 2-5). Next, when the substrate is exposed to EM radiation, the release of energy destroys the magnetic pattern, either because of mechanical damage or because of demagnetizing the media by rising temperature in hybridized spots.

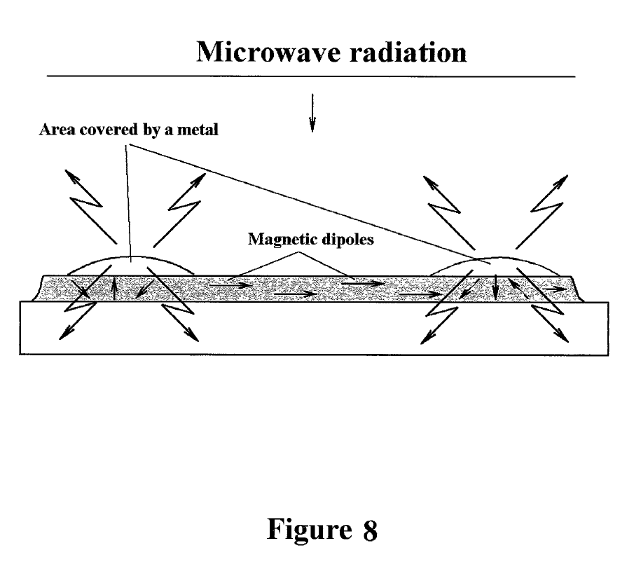

The current state of the pattern on recording media can be analyzed by reading the record and by comparing what was actually read with what was recorded at the same spot of the media. Errors or disturbance of the magnetic pattern would point to the spots where probe and target were hybridized.

FIG. 9 shows an another way for monitoring hybridization of target and probe species by using “Reaction” and “Witness” plates. The surface of the Reaction plate is used for hybridization of the probe and target species. The spots of the Reaction plate where probe and targets are hybridized next is covered by a reporter material, for example, by using biotinylated targets and streptavidin “functionalized” reporter particles. After tagging with the reporter material, the Reaction plate is placed in close mechanical contact with the Witness plate, which has a surface covered or painted by a sensitizing material. The assembly of the Reaction and Witness plates is exposed to a microwave radiation. A process initiated by microwave radiation on the interface of the Reaction and Witness plates types the spots from Reaction plates onto the sensitive layer of the Witness plate. Therefore, the pattern of spots from the Reaction plate can be found by monitoring the prints on the surface of the Witness plate.

FIG. 10: Example of using 25 nm gold particles to produce physical damage on the surface of a magnetic floppy disk. (A) is the surface of the disk with gold particles deposited from colloid solution and seen on the left side of the image. (B) the same surface as in (B) after it was exposed by microwave radiation. “Radiation marks” points to the spots where the surface was damaged by the release of microwave energy.

FIG. 11: (A) A commercial CD-Recordable disk with protective layer removed to provide the access to the disk sensitive surface; (B) metal particles on the surface of the CD-R disk prepared for burning pits by exposing to microwave radiation.

FIG. 12: Snapshot of computer screen of the system for analyzing physical state of recording media. Red spots mark locations where damage of sensitive layer of the disk was detected.

### DESCRIPTION OF THE PREFERRED EMBODIMENTS

The present invention is based on the observation that some materials, such as, but not limited to metals, can be bound to a surface by a highly controllable way and, said materials, then can be used to trigger substantial release of energy on the surface when the surface and its content are exposed to EM radiation, including, but is not limited to, microwave radiation. Depending on the intensity of microwave radiation and properties of the surface and said material, the effect from release of the energy can vary from local over-heating of the surface to micro-explosion (arcing) and even ejecting substance from the surface. Spatial size of the area on the surface affected by the release of EM radiation energy might be significantly bigger than the size of the area initially covered by the material which has triggered the process. Therefore, the change on the surface, which resulted from the release of EM radiation energy, can be used to point out the location on the surface and quantitatively characterize the amount of said material to provide information about the primary process which bound radiation absorbing material to the surface.

In particular, we have discovered that biomolecules, such as oligonucleotides or proteins, bound to a surface and tagged with metal particles can then be detected, and the location of the biomolecules on the surface can be identified by exposing the surface and its content to an electromagnetic radiation, and particularly, to microwave radiation, and more specifically, through the steps of:

1. Solid surface, on which analysis will be performed, first is prepared by covering or by painting the surface with a thin layer of a material (paint) with a distinguishable property, i.e., either of an optical property, mechanical property, magnetic property, or chemical property. Said surface might be a surface of a plate of glass, plastic, or any other material which does not have significant absorption of EM radiation, which said radiation is used for treatment as described herein. Said plate might have any shape and size including, but not limited to, the rectangular shape or might be shaped as a disk, similar to a computer compact disk (CD) or computer floppy disk.

A microarray of probe oligonucleotide or proteins is prepared on said surface by binding the species to the surface the way it is described in the previous art, see, e.g., Gingeras, et al., “Hybridization Properties of Immobilized Nucleic Acids”, Nucleic Acids Res., 15(13), 5373-5390 (1987); Saiki et al, “Genetic Analysis of Amplified DNA with Immobilized Sequence-Specific Oligonucleotide Probes”, Proc. Natl. Acad. Sci. USA., 86, 6230-6234 (1989); Chee et al, “Accessing Genetic Information With High-Density DNA Arrays”, Science, 274, 5287 (1996); Cheung et al, “Making and Reading Microarrays”, Nature Genetics, 21(1), 15-20 (1999); Lipshutz et al, “High Density Synthetic Oligonucleotide Arrays”, Nature Genetics, 21(1), 20-25 (1999). The cited art is hereby incorporated herein by reference so that the general procedures and methods in that art that are of use to practice of the present invention need not be rewritten herein. Location on the surface of each specific type of probe is known and the location of the probe can be used to uniquely identify type of the specie, for example, based on its sequence in the case of oligonucleotide, or based on sequence and/or structure in the case of proteins.

2. Said surface with immobilized probes is exposed to solution of target species, which would be bound or hybridized to the probes on the surface if said target is complementary to the probe because of the primary sequence. (For example, in the case of oligonucleotides, or structure, in the case of proteins.) The targets do not bound to the surface if they are not complementary to the probes on the surface, see, e.g., Dale (2000) U.S. Pat. No. 6,087,112; Hori et al., (2001), U.S. Pat. No. 6,194,148; Fodor et al., (2001) U.S. Pat. No. 6,197,326; Fodor et al., (1992), Pat. No. WO92/10588; Virtanen, (1998), Pat. No. WO98/01533. The target species have the capability of attaching reporter molecules or particles through the reaction similar to biotin-streptavidin reaction, thioether linkage , or other methods of covalent or non-covalent molecule-surface binding known from the previous art, see, e.g., Forster et al, “Non-Radioactive Hybridization Probes Prepared by the Chemical Labeling of DNA and RNA with a Novel Reagent, Photobiotin”, Nucleic Acids research, 13(3), 745-761 (1985); Symons et al., U.S. Pat. No. 4,898,951; Lavrich et al, “Physiosorption and Chemisorption of Alkanethiols and Alkylsulfides on Au(111)”, Princenton University, Princeton, N.J. 08544; Hegde et al., “A Concise Guide to cDNA Microarray Analysis”, Biotechniques, 29, 548-562 (2000)

3. The targets, which were not bound or hybridized to the probes on the surface during Step 2, are washed away and the surface and bound species are exposed to the solution of reporter material, either molecules or particles, including either micro- or nano-meter size particles. The material for the reporter species is chosen from a set of materials which can efficiently interact and/or absorb electromagnetic radiation. Such materials might include, but are not limited to, pure metals, metal alloys, metal compounds, semiconductors, etc. The reporter particles are attached to the surface in locations where target and probe have been bound or hybridized.

4. When necessary, the surface with immobilized tagging particles can be additionally treated by confining said surface substrate between two smooth solid surfaces (casts) and by applying pressure to the casts of usually not less than 10⁵ Pa, which of capable to squeeze said substrate and the tagging particles on said substrate surface to the degree when the tagging particles would penetrate or immerse into the sensitive layer of said substrate. Such treatment can improve the mechanical contact between tagging particles and solid substrate.

5. Substrate with species on its surface tagged by the reporter is placed for treatment into a microwave oven similar or identical to a consumer microwave oven. Equally acceptable, said substrate can be treated by any other source of electromagnetic radiation, including, but is not limited to a source of coherent laser radiation, whereby said source is capable to produce radiation which can be absorbed/dissipated by tagging particles. Next, the sample is exposed to an electromagnetic radiation. The exposure time can vary from seconds to minutes depending on which property of the substrate and reporter particles is used. Extensive release energy of EM radiation in the spots modifies or damages the underlying layer of the substrate, which was sensitized as was described in the Step 1. The size of the area where the substrate coverage was affected by EM radiation might be significantly bigger than the area originally covered by a reporter material. This, in fact, increases visibility of the small amount of reporter material on the surface and makes it possible to detect the location on the surface and measure the quantity of the reporter material. Since the reporter material would be allocated only in spots where probe and target were bound or hybridized, the modification of the substrate surface by EM radiation indicates the spots where the probe is complimentary to the target. Therefore the sequence or structural information about target can be obtained for DNA and protein microarrays respectively.

6. Yet in the another embodiment of the invention, we discovered that the solid surface, on which analysis will be performed, first can be covered or painted by a thin layer of a magnetic paint, which is similar or identical to one used in manufacturing computer floppy disks, see, e.g., J. U. Lemke, “Magnetic Storage: Principles and Trends”, MRS Bulletin, March 1990, pp.31-35; M. P. Sharrock, “Particulate Recording Media”, MRS Bulletin, March 1990, pp.53-61; and J. H. Judy, “Thin Film Recording Media”, MRS Bulletin, March 1990, pp.63-72. Said surface might be a plastic disk similar or identical to a computer floppy disk. Before performing steps 2-4 as described above for obtaining sequence or structural information on target moieties, the surface of the disk can be magnetized or special magnetic pattern can be recorded on the disk essentially through the same steps used for recording digital information on floppy disks. Then steps 2-4 can be pursued as described herein. Release of EM energy on the surface of the magnetic paint can destroy the magnetic pattern recorded on the disk, either because of producing mechanical damage of the surface or because of demagnetizing the magnetic material in the spots where reporter material was bound to the surface. We would like point out here that magnetizing and demagnetizing magnetic material in the spots due to rising temperature is known, for example, from technology of magneto-optical computer disks, or from the approach of “thermo-coping” magnetic audio and video records when chromium based magnetic media is used. The condition of the magnetic layer and magnetized pattern can be analyzed by reading back the magnetic pattern and by comparing what was read with what was recorded at the same spot on the disk. Read errors, i.e., the discrepancy in the pattern read versus the pattern that has been written, would indicate the spots where the reporter material was bound to the surface, and thus, would indicate the location where probe and target were hybridized. It is essential that in this embodiment of the invention the target-probe bond or hybridization can be detected even when the underlying array's surface stays mechanically intact. The hybridization events still can be detected because of complete or partial destruction of the magnetic pattern on the array's surface.

7. Yet in the another embodiment of the invention, we also discovered that the solid surface, on which analysis will be performed, first can be covered or painted with a concentric pattern of tracks in a way very similar or identical to that used for manufacturing of a recordable compact disk (CD). Said surface might be a plastic disk similar or identical to a computer compact disk (CD). Before performing steps 2-4 as described above for obtaining sequence or structural information of the target moieties, an optical properties of the tracks on the surface can be modified by burning a pattern, which is performed similar or identical to the way information is written to computer compact disk (CD). Then steps 2-4 can be pursued as described above Dissipation of electromagnetic energy on the surface of the paint can destroy the tracks and the pattern burned on the disk mainly by means of producing mechanical damage or because of modifying optical properties of the paint in the spots where reporter material was bound to the surface. The condition of the tracks and recorded pattern can be analyzed by reading back the pattern and by comparing what was read with what was recorded at the same spot on the disk. Read errors, i.e., the discrepancy of the pattern read versus the pattern that has been written, would indicate the spots where the reporter material was bound to the surface, and thus, would indicate the location where probe and target moieties were hybridized.

8. Yet in another embodiment of the invention, we found the steps 2-4 can be pursued using a plate, referred as a Reaction plate, with a surface which was not treated as described in Step 1. To detect spots where probe and target were bound or hybridized and where the reporter material was bound to the surface plate, the Reaction plate after pursuing step 4 is placed in close mechanical contact with another plate, referred as Witness plate, which was treated as described in the Step 1 above, but which did not go through the Step 2-4. The assembly of the Reaction and Witness plates then is put for treatment by a source of EM radiation, such as, for example, a microwave oven. Extensive release energy of EM radiation in the spots where the reporter material is bound to the surface of the Reaction plate modifies or damages the layer of paint of the Witness plate, which is in close mechanical contact with the Reaction plate. Therefore, the procedure enables transfer of the pattern from the Reaction plate with the spots of the reporter material onto the surface of the Witness plate. Later the Witness plate can be analyzed to find out spots where the probe and target moieties were hybridized on the Reaction plate. An important aspect of this embodiment of the invention is that two different plates are used for monitoring binding or hybridization, such that the surface of one plate, the Reaction plate, can be optimized for attachment probes and for hybridization, and the surface of the another plate, the Witness plate, can be optimized for efficient detection of a small amount of the reporter material. The monitoring of the Witness plate can be done using the techniques including, but not limited to, magnetic or optical detection as known from the previous art and was disclosed herein.

### EXAMPLES

The following section presents particular examples of implementation of the system covered by this invention. However, possible design of the system is not limited to these particular examples. The disclosure presented herein enables one of average skill in the art to practice the present invention in many different forms to achieve a desired analyte or particle detection capability to suit many others diagnostic assay format types, and apparatus types. Different types of inexpensive apparatus and test kits can be made by practice of the invention in one form or another to suit a specific analytic diagnostic need.

### Example

### Microwave Treatment for Enhancing Detection Sensitivity

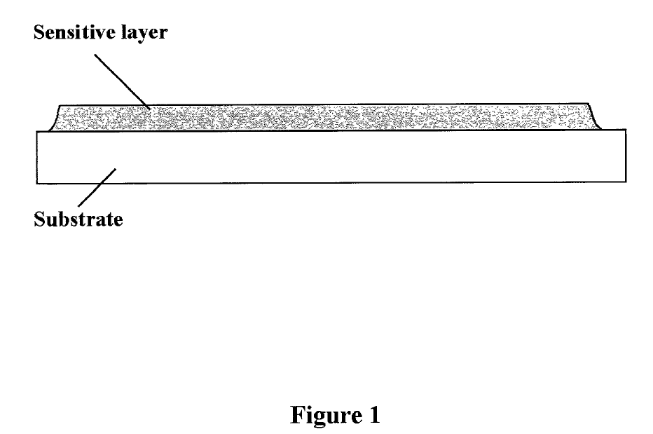

An objective of this particular example is to overcome some current limitation of sensitivity and selectivity of DNA microarrays. In this example, highly sensitive detection of DNA hybridization on a surface can be achieved by amplifying a small change of a local property of the surface at the spot where hybridization has occurred. The array surface can be monitored and hybridization on the surface can be qualitatively and quantitatively characterized by using inexpensive and highly developed technology based on an optical reader similar to a computer CD reader. For an outline of relevant prior art see, e.g., Wang et al, (1999), U.S. Pat. No. 5,922,617; Adelman, (1997), U.S. Pat. No. 5,656,429; Virtanen, (2001), U.S. Pat. No. 6,200,755; Gordon et al., (1996), Pat. No. WO96/09548; Demers, (1998), Pat. No. WO98/12559; Virtanen, (1998), Pat. No. WO98/38510; and Remacle, (1999), Pat. No. WO99/35399. By using approach disclosed in our present invention the sensitivity of detection of biopolymer molecules can he further improved versus approaches and techniques known from the previous art. An overview of steps for preparing the surface of a CD microarray for hybridization detection and using EM radiation treatment for enhancing detection sensitivity are presented in steps illustrated in FIGS. 1-5. More details are described as follows:

a. A writable CD is used as a substrate for preparing DNA array. The CD surface is covered by a thin layer of a “witness” material , i.e, a layer of water-non-soluble dye which has strong optical absorption for detection. The witness material can cover the surface uniformly, or it can be deposited with a pattern of concentric tracks on a disk surface depending on the kind of equipment used to analyze the surface.

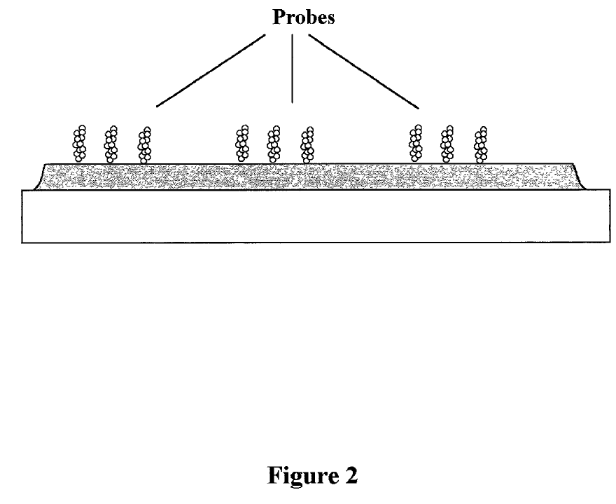

b. As illustrated in FIG. 2, probes are immobilized on small spots of the surface, such that each spot contains probes with a specific sequence and the location on the surface of each particular probe is known (see FIG. 2). A number of methods and commercial kits are available to link DNA to the surface. As an example, in the protocol from Brown Lab of Stanford University, the array surface is first covered by polylysine, following with rehydration, printing probes and UV crosslinking. Some other protocols include aldehyde coating for the direct attachment of DNA to the surface, and using epoxysilynated surfaces to tether DNA containing amino linkages at its termini.

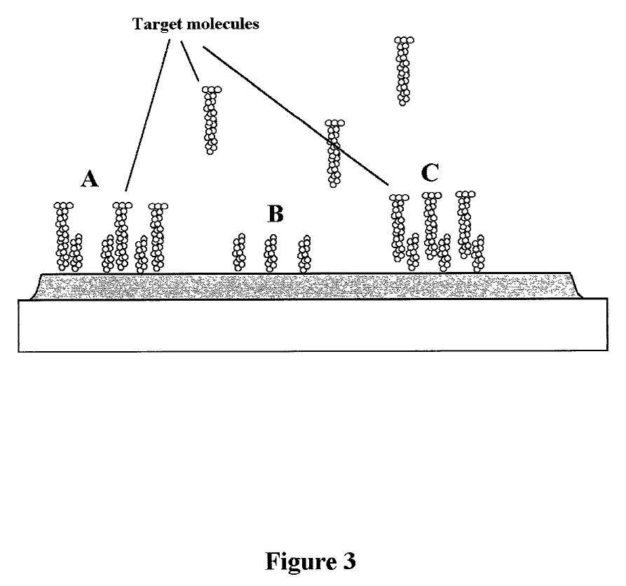

c. Before performing hybridization with probes, genomic target DNAs or PCR products are labeled with biotin. It is equally acceptable to use either terminal biotin labeling during the PCR process or the photoactivable form of biotin for covalent attachment to nucleic acids as described by Forster et al, “Non-Radioactive Hybridization Probes Prepared by the Chemical Labeling of DNA and RNA with a Novel Reagent, Photobiotin”, Nucleic Acids research, 13(3), 745-761 (1985); see also Symons et al., U.S. Pat. No. 4,898,951. Once the target DNA is prepared, the array surface is exposed to the solution of the biotinylated target DNA. Then target and probe molecules are hybridized on spots where probe and target have complementary sequence as illustrated in FIG. 3.

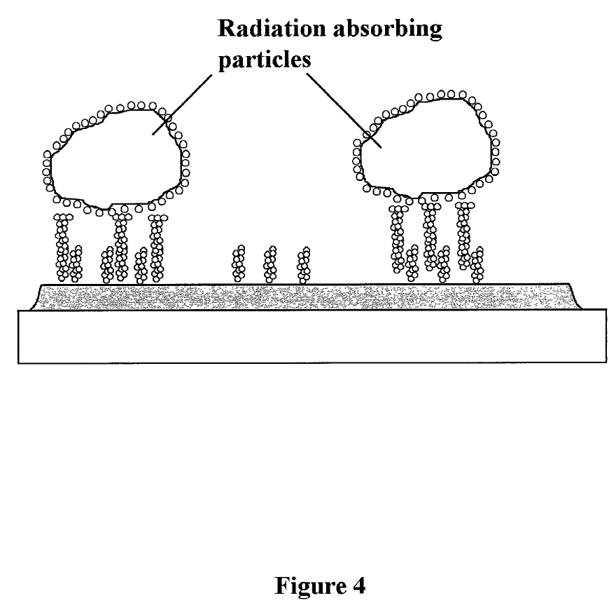

d. Array surface is exposed to a colloid solution of streptavidin coated metal particles. Micro-size metal particles with streptavidin on the surface are currently commercially available from a few vendors. During this step, metal particles are attached to the array's surface on spots where probes and biotinylated targets are hybridized.(See FIG. 4) To remove non-specifically hybridized molecules, the array can be washed at the temperature just 1-2 degrees below the optimal stringency temperature. This step is expected to be especially efficient for metal tagged hybridized complexes. It was discovered recently, tagging by a metal alters the melting profiles of the hybridized probe and target DNAs, see, e.g., Taton et al, Scanometric DNA Array Detetction with Nanoparticle Probes”, Science, 289, 1757-1760 (2000) and references herein. The difference permits better discrimination between perfectly matched and mismatched hybridization and therefore provides a unique opportunity to improve selectivity. Attachment of metal particles to hybridized DNA complexes provide advantages in delivering a desirable amount of reporter material per single DNA complex, as compared with conventional fluorescent tagging. Indeed, consideration of mechanical strength of the probe-target pair indicates that even single complex is able to anchor a 1 um size particle on the array surface. The amount of tagging material can be of 7×10⁻¹² g versus the mass of a single fluorescent molecule of 2×10⁻²² g .

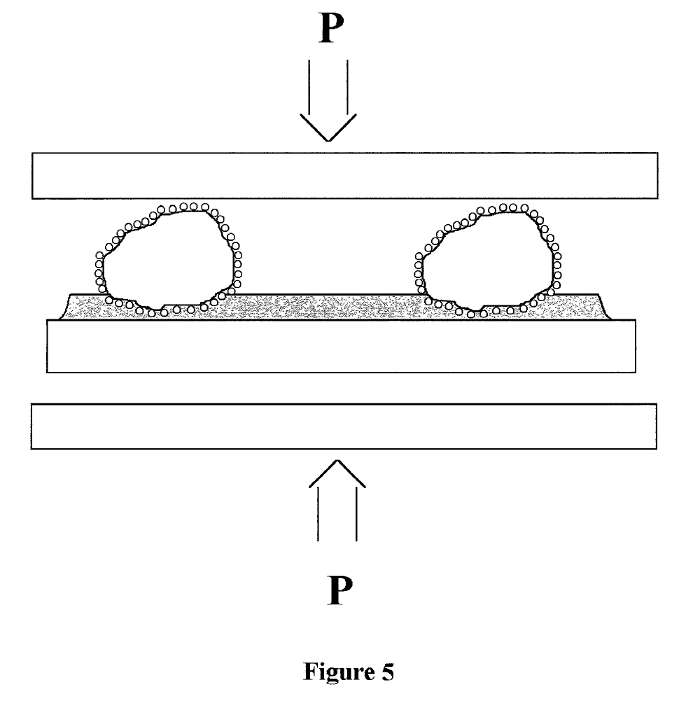

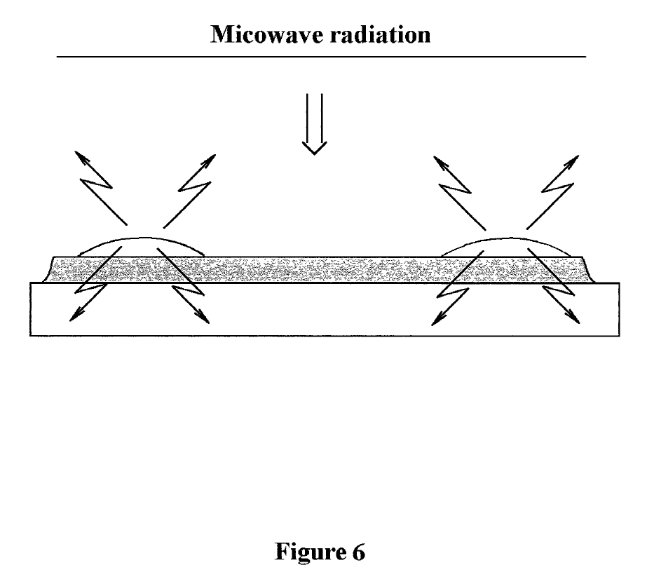

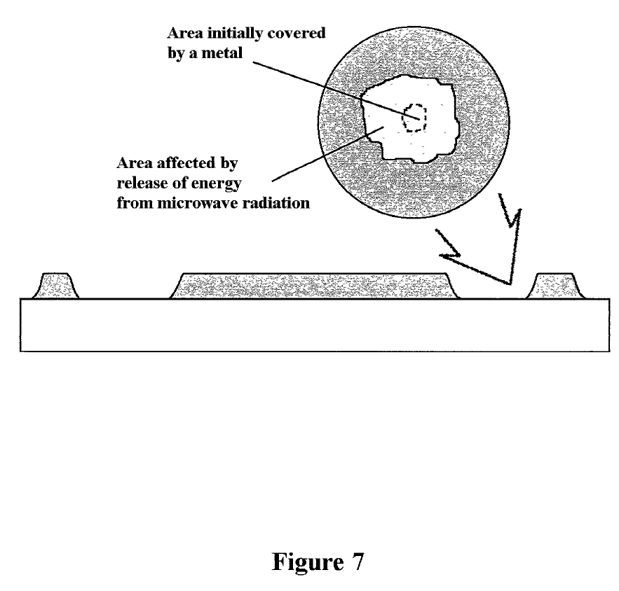

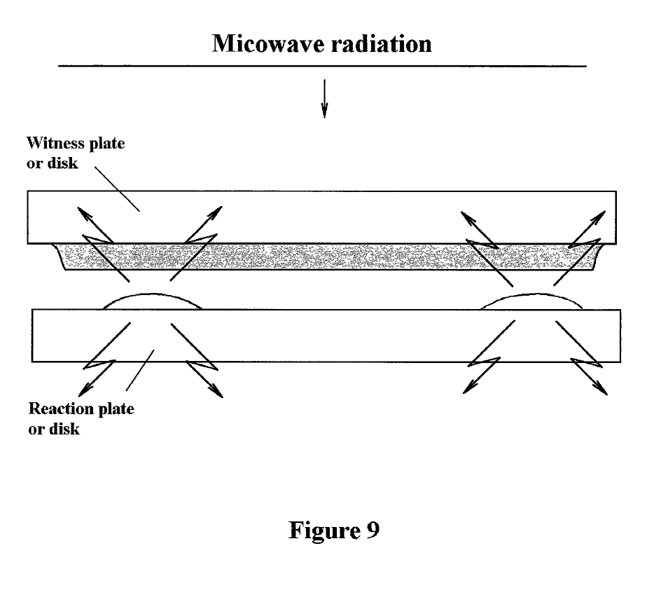

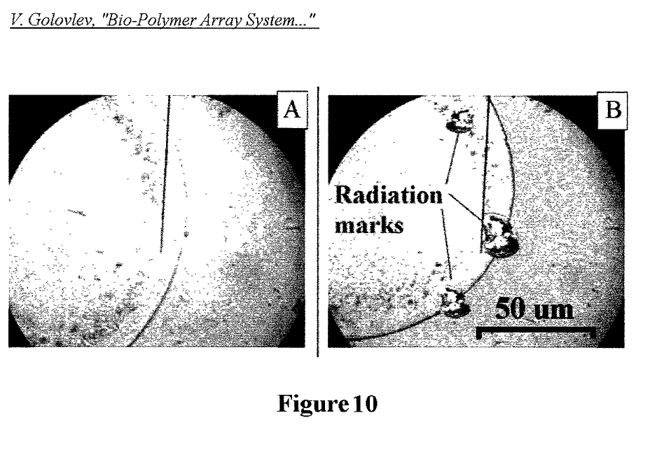

When metal particles immobilized on dielectric substrate are exposed to EM radiation, and particularly to microwave radiation, the energy absorbed by metal particles can be significantly higher than the EM energy absorbed and released by a dielectric substrate. Fast energy release in metal causes its overheating and explosive evaporation which “bums” and damages the surface area much bigger than the area originally covered by a metal. FIGS. 5, 6, and 7 illustrate this process schematically and FIG. 9 shows that the EM radiation heating can produce the mark from the sample on one plate to the other. FIGS. 10 A,B shows a photograph of the actual effect of microwave radiation on the spot covered by gold particles. FIG. 10A shows the spot covered by gold particles deposited on the substrate by drying colloid solution of 25 nm size particles. A total amount of gold in the spot in FIG. 10A can be estimated as 10⁻⁸ g. The particles have effectively covered an area of 1000 sq. um, thus the density of the gold coverage is estimated as 10⁻¹¹ g/sq. um.

The substrate was then exposed to microwave radiation; the volume density of radiation in the microwave cavity was 10 kW/m³. The exposure time was of 10 sec. FIG. 9B shows the picture of the surface taken after the surface was exposed by microwave radiation. Marks in FIG. 9B indicate damage spots were found only in the area covered by gold particles. The initial size of “nucleus” where explosive evaporation have occur can be estimated as about 1 sq.um, and therefore the amount of metal material required for easily detectable damage on the surface at the present experimental condition is of 10⁻¹¹ g. In this experiment 25 nm gold particles were used to deposit gold material on the surface. The mass of an individual colloid particle estimated from its size and the density of gold is 25 nm×25 nm×25 nm×12.500 g/cm³=10⁻¹⁶ g, and one can estimate that the number of gold particles in a single cluster which initiated the damage on the surface is about 3×10⁵. This number corresponds to the number of DNA molecules in the local spot on the surface to be detected. The amount is equal to about 0.0005 femto Mole. This sensitivity is two to three orders of magnitude better than fluorescence detection of DNA and also significantly more sensitive than radioactive tagging method.

Further increase of sensitivity can be achieved by increasing the intensity of the microwave radiation and by increasing the size of the metal particle used for tagging. We expect that optimization of the experimental condition can increase the sensitivity by another one to two order of magnitude, compared with what has been presented here. In principle, there is a potential to even detect single DNA hybridization. With the improvement of sensitivity, the use of PCR may not be needed. It will save significant time for DNA analysis. It can also be used to probe genomic DNAs.

### Example

### Array Reading System

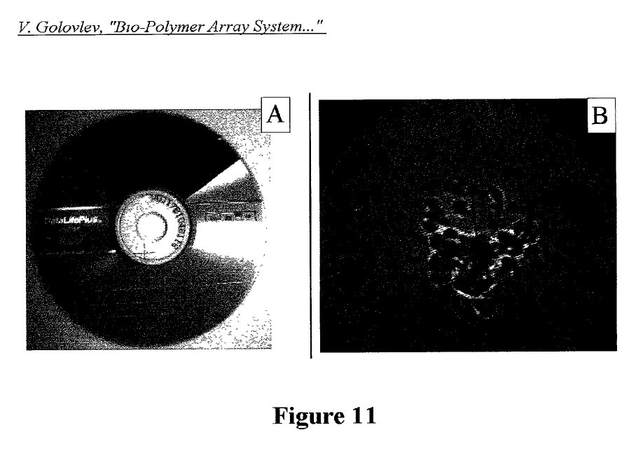

To make microarray reading less expensive and easy to use in the field, in this particular example of implementation of the invention, we will take the benefit of know-how developed in the computer field to prepare DNA array on disks similar to optical compact disk (CD) and use a commercial computer CD drive as a platform for reading the array. Preparation and use of a standard CD disk in such an experiment is illustrated in FIG. 11. A standard commercial recordable CD-R is a plastic 5″ disk assembled as a sandwich of polycarbonate substrate with dye recording layer, reflective metallic film, and protective layers on top of it. Information is recorded on CD-R using a laser to burn pits in the organic dye. Photochemical decomposition of dye on the surface of the disk produced during the recording phase changes optical properties. It can be detected and read during the reading phase, when the laser beam is tightly focused onto the recording surface of the disk which is in contact with the reflective layer. To use a standard commercial disk as a substrate for DNA array, the protective layer needs to be removed as shown in FIG. 11 to provide access to the recording surface of the disk. The surface then can be treated and the DNA probe can be attached to the surface using known DNA's microarrays protocols. After hybridization and tagging DNA with metal particles, the disk is exposed to microwave radiation, which causes explosive evaporation of the metal clusters on the surface and produces damage of the organic dye on the recording surface that is very similar the way a laser produces photo-chemical decomposition of dye during the recording phase. The disk can be read by a standard CD reader and the area where probe and metal attached target DNA hybridized will be recognized because of the change of the optical property of the disk.

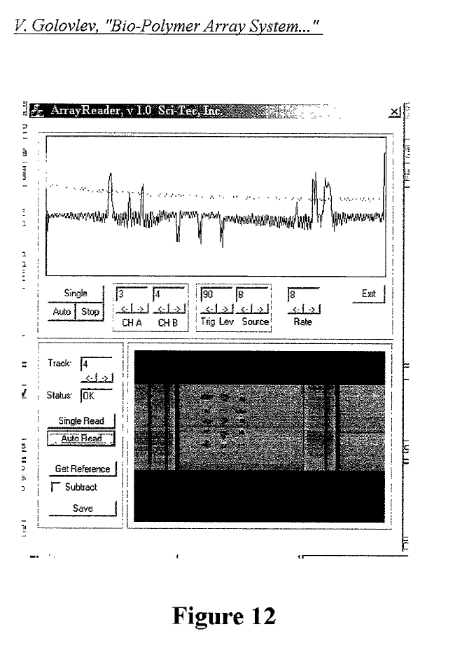

The reading noise can be reduced significantly if, before using the CD for DNA detection, the disk is formatted by recording a reference pattern using a standard CD writer. The pattern recorded on disk might be a file, for example, with a continuous set of 0 and 1:01010101. . . The pattern provides a reference set for the comparison of what was written and what was actually read from the same spatial location on the disk. Read errors are generated in spots where recording media was damaged by explosive evaporation of metal particles. The errors mark spots where probe and target DNA were hybridized. FIG. 12 shows a snapshot of a computer screen using our experimental system to analyze the surface of recording media. The top view window shows an analog signal acquired from an individual track of the disk and the bottom view window shows a map representation of 1 cm×1 cm area of the disk with spots marking the surface on the disk where recording error was detected. The spots in the bottom view window in FIG. 12 marks defects on the disk, which were created by directly depositing non-magnetic metal material on the disk's surface. Similar approach and system can be used to analyze surface of the magnetic media, such as magnetic diskette, where said media and its surface can be used to carry an array of probes for hybridization analysis.

What is claimed is:

1. A process for identifying the presence or absence of a specific sequence in a target bio-polymer molecule, for the purpose of sequencing, fingerprinting, or mapping said target bio-polymer molecules, typically biological polymers, comprising the steps of:
a) preparing a surface on which analysis will be performed by covering a solid substrate with a thin layer of material, also referred to as sensitive layer, with distinguishable properties, such as mechanical, optical, magnetic, or chemical property;
b) preparing multiple test sides on said surface by immobilizing probes. The probes will be of various known structures selected to bind with molecular structures, such as biological polymers, which may be in the sample of being analyzed. Location and type of each particular probe on the surface is known and therefore probe location on the surface can be used to identify type/sequence of the probe;
c) hybridizing a target bio-polymer molecule with a bio-polymer probe on said surface, which said probe is complementary to a region of the target molecule;
d) labeling/tagging the hybridized probe-target complexes with a metal particle, or particles of other material, which are capable, if present, of absorbing and dissipating electromagnetic radiation (EM), which said EM radiation means either microwave radiation, infra-red radiation, visible or ultra-violet light;
e) exposing said surface with labeled probe-target complexes by electromagnetic radiation under conditions where the energy of the radiation can be dissipated by said tagging particles. Dissipation of electromagnetic energy by tagging particles causes change of local properties of said sensitive layer on said surface in spots where probe-target complexes are located;
f) analyzing said surface by measuring property of the sensitive layer, using appropriate technique(s) for measuring mechanical, optical, magnetic, or chemical parameters of said sensitive layer as known from prior art. Deviation or change of the quantitative characteristic of the sensitive layer resulting from EM radiation exposure would point to the spots where probes were hybridized to the targets, therefore pointing to the presence of targets with a sequence complimentary to the sequence of corresponding known probe(s);

2. A process of claim 1, wherein said surface might be a plate of glass, plastic, or any other material which does not have significant absorption of electromagnetic radiation in the absence of tagging particles. Said plate might have any shape and size including, but not limited to, the rectangular shape or might be shaped as a disk, similar to a computer compact disk (CD) or computer floppy disk.

3. A process of claim 1, wherein the sensitive layer is a layer of magnetic material similar to that used in computer magnetic media, such as computer floppy disks.

4. A process of claim 1, wherein the sensitive layer is a layer of material capable of absorbing, reflecting or scattering light from an external light source. Said material is similar to that used in compact laser disks such as computer CDs.

5. A process of claim 1, wherein the surface on which analysis will be performed is shaped as a disk similar to a computer floppy disk or computer CD.

6. A process of claim 1, wherein labeling/tagging particles consists essentially of a metal or metal alloys such as, but are not limited to, gold, silver, gold alloys and silver alloys.

7. A process according to claim 5, wherein labeling/tagging particles have the size from 5 nm to 5 um, usually not less than 1 nm, and most preferably not bigger than 10 um.

8. A process of claim 1, wherein the substrate with immobilized tagging particles, prior of being exposed to EM radiation, is treated by confining said substrate between two smooth solid surfaces (also referred as casts) and by applying pressure to the casts of 10⁸ Pa, usually not less than 10⁵ Pa, and most preferably not more than 5×10⁹ Pa; Said pressure is of capable to squeeze said substrate and the tagging particles on the substrate surface to the degree when the tagging particles start to penetrate or immerse into the sensitive layer of said substrate; The treatment, as disclosed herein, can improve the mechanical contact between tagging particles and solid substrate.

9. A process of claim 1, wherein exposure to electromagnetic radiation means exposure to microwave radiation; said exposure can be performed by a device similar to or identical to a consumer microwave oven.

10. A process of claim 1, wherein exposure to electromagnetic radiation enhances detection sensitivity by producing physical or chemical effects on the surface with immobilized tagging particles therefore making more “visible” the presence of a small amount of tagging material immobilized on a surface by probe-target complexes.

11. A process according to claim 2, wherein the surface is analyzed by detecting magnetic response of the magnetic material on the surface using the device similar to a computer floppy drive.

12. A process according to claim 3, wherein the surface is analyzed by detecting optical response of the sensitive layer using approach and device similar to a computer CD drive.

13. A process of claim 1, wherein exposure to electromagnetic radiation can be performed using a coherent laser radiation.

14. A process of claim 8, wherein the treatment by EM radiation is void; After casting of said substrate as disclosed herein by claim 8 the surface of the substrate is analyzed by monitoring change of mechanical, optical, magnetic, or chemical parameters of said sensitive layer of the substrate which said changes are resulted from penetration or immersing of tagging particles into the sensitive layer of the substrate.

15. A process for identifying the presence or absence of a specific sequence in a target bio-polymer molecule, for the purpose of sequencing, fingerprinting, or mapping said target bio-polymer molecules, typically biological polymers, using Reaction and Witness plates and comprising the steps of:
a) preparing a substrate on which analysis will be performed, also referred as a Reaction plate, by preparing multiple test sides on said substrate surface by immobilizing probes. The probes will be of various known structures selected to bind with molecular structures, such as biological polymers, which may be in the sample of being analyzed. Location and type of each particular probe on the surface is known and therefore probe location on the surface can be used to identify type/sequence of the probe;
b) hybridizing a target bio-polymer molecule with a bio-polymer probe on the surface of said Reaction plate, which said probe is complementary to a region of the target molecule;
c) labeling/tagging the hybridized probe-target complexes with a metal particle, or particles of other material, which are capable, if present, of absorbing and dissipating electromagnetic radiation, which said radiation means either microwave radiation, infra-red radiation, visible or ultra-violet light;
d) preparing a surface of another plate, which will be referred as a Witness plate, by covering a solid substrate with a thin layer of material, also referred to as sensitive layer, with distinguishable properties, such as mechanical, optical, magnetic, or chemical property as was disclosed herein;
e) placing said Reaction and Witness plate in close mechanical contact, such that surface of the Reaction plate with immobilized tagging particles is being in contact or is being in very close proximity to the sensitive layer of the Witness plate; the Reaction and Witness plate therefore are forming a sandwich like structure;
f) exposing said sandwich of the Reaction and Witness plates by electromagnetic radiation under conditions where the energy of the radiation can be dissipated by said tagging particles on the interface of the Reaction and Witness plates. Dissipation of electromagnetic energy by tagging particles causes change of local properties of said sensitive layer of the Witness plate in spots where probe-target complexes are located;
g) analyzing said surface of the Witness plate by measuring property of the sensitive layer, using appropriate technique(s) for measuring mechanical, optical, magnetic, or chemical parameters of said sensitive layer as known from prior art. Deviation or change of the quantitative characteristic of the sensitive layer resulting from EM radiation exposure would point to the spots where probes were hybridized to the targets, therefore pointing to the presence of targets with a sequence complimentary to the sequence of corresponding known probe(s);

16. A process of claim 15, wherein said Reaction and Witness plates might be a plate of glass, plastic, or any other material which does not have significant absorption of electromagnetic radiation in the absence of tagging particles. Said plate might have any shape and size including, but not limited to, the rectangular shape or might be shaped as a disk, similar to a computer compact disk (CD) or computer floppy disk.

17. A process of claim 16, wherein the sandwich of the Reaction and Witness plates, prior of being exposed to EM radiation, is treated by confining said sandwich of Reaction and Witness plate between two smooth solid surfaces (also referred as casts) and by applying pressure to the casts of 10⁸ Pa, usually not less than 10 ⁵ Pa, and most preferably not more than 5×10⁹ Pa; Said pressure is of capable to squeeze said Reaction and Witness plates and the tagging particles on the interface of the Reaction and Witness plates to the degree when the tagging particles start to penetrate or immerse into the sensitive layer of the Witness plate; treatment, as disclosed herein, can improve the mechanical contact between tagging particles and sensitive layer of the Witness plate;

18. A process of claim 17, wherein the treatment by EM radiation is void; After casting of said sandwich of the Reaction and Witness plates as disclosed herein by claim 17 the surface of the Witness plate is analyzed by monitoring change of mechanical, optical, magnetic, or chemical parameters of said sensitive layer of the substrate which said changes are resulted from penetrating or immersing of tagging particles from the surface of Reaction plate into the sensitive layer of the Witness plate.
